(19) United States
(12) Patent Application Publication
(10) Pub. No.: US 2025/0057438 A1
(43) Pub. Date: Feb. 20, 2025

(54) SYSTEM AND METHOD OF SENSING CATHETER'S LOCATION AND FORCE

(71) Applicant: BIOSENSE WEBSTER (ISRAEL) LTD., Yokneam (IL)

(72) Inventors: Nathanel Yehuda David Hayat, Petah Tikva (IL); Barry Neal Breen, Givat Zeev (IL); Daniel Zohar, Zichron Yakov (IL); Avraham Cohen, Kiryat Bialik (IL)

(21) Appl. No.: 18/807,145

(22) Filed: Aug. 16, 2024

Related U.S. Application Data
(60) Provisional application No. 63/533,258, filed on Aug. 17, 2023.

(51) Int. Cl. A61B 5/06; G01R 33/00

(52) U.S. Cl. CPC A61B 5/062; G01R 33/0005; G01R 33/0052; A61B 2562/0223; A61B 2562/12

(57) ABSTRACT

An electric component having electric coils, the component formed by 3D printing of at least three different 3D-printed materials with spatial distributions yielding open magnetic circuit configuration of one of the electric coils. The electric component having a bulk formed by non-magnetic and dielectric 3D printed material; and an electric coil of the open magnetic circuit configuration, 3D-printed in the bulk. A magnetic channel of magnetic material 3D-printed in the bulk forming magnetic core of the electric coil; and an electric channel forming an inductor of the electric coil having conductive material 3D-printed in the bulk with a coil/helical geometry having electrically connected conductive windings arranged to circumference the magnetic channel, which is configured and operable with the open magnetic circuit configuration and has magnetic material occupying a central region of the coil/helical geometry of the inductor while not enclosing the windings with a closed loop of the magnetic material.

### CROSS-REFERENCE TO RELATED APPLICATION

This application claims the benefit of U.S. Patent Provisional Application 63/533,258, filed Aug. 17, 2023, the contents of which are incorporated by reference as if set forth in its entirety herein.

### TECHNOLOGICAL FIELD

The present invention is in the field of catheters and particularly relates to systems and methods for sensing catheter's location and the force applied thereby.

### BACKGROUND

In various therapeutic and diagnostic procedures, a catheter/probe is inserted into a patient's body (e.g., chamber of the heart) and to be brought into contact with a body tissue there. Typically, in such procedures, it is necessary to determine the catheter's location within the body (i.e., the location at which a distal tip of the catheter engages the body tissue) as well as the pressure applied thereby to the tissue.

Catheters having integrated location and pressure sensors for sensing the location of the catheter and the pressure/force applied thereby at the contact region with the tissue, are generally known. Such catheters typically utilize inductive coils for determining the location of the catheter within the body and/or the pressure/force applied thereby to a body tissue it engages with.

Conventional techniques for such catheters often utilize wire-winded coils to which a ferrite core may be manually inserted after the winding. The coils fabricated in this way are then soldered to a cable that carries the coil signal to processing circuits, and fitted within the catheter's body.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A is a flow diagram of a method 100 according to an embodiment of the present invention for fabricating electrical components with one or more open magnetic circuit electric coils therein;

FIGS. 1B to 1D are respectively a perspective view, several lateral cross-sectionals views and a side-view cross-section, of a model 200 of an example electric component 210 fabricated by the technique of the present invention;

FIG. 1E, is schematic perspective view illustration of two example electric components 210(a) 210(b) of the present invention, which are suitable for use as medical instrument sensors;

FIGS. 1F and 1G, are schematic perspective view illustration of six example electric components 210(c) to 210(h) according to the present invention, each including a single open magnetic circuit coil 3D-printed by the technique of the present invention; and

FIG. 2 shows a side-view of a medical instrument 1000 such as a catheter according to an embodiment of the present invention, in which electric components 210 of the present invention are furnished as position and force sensors.

### DETAILED DESCRIPTION OF EMBODIMENTS

There is a need in the art of catheters, for physically agile and sensitive catheters having compact/narrow dimensions, that are capable of providing accurate feedback on the location of the catheter in the body, and the contact interface between the catheter and the tissue and the force (e.g., vector) applied between them.

U.S. Pat. No. 8,535,308 and U.S. patent application No. U.S. 2020/015693, which are both assigned to the assignee of the present application, disclose for example configurations of such catheters utilizing sensory circuits having several coils, for sensing the location and orientation of the catheter body, as well as the force applied thereby at the interface with the tissue.

One challenge in the art of such catheters is to fit accurate location and force sensors within the narrow body of the catheter, whose lateral dimensions are typically only several millimeters. Indeed, as indicated above, in conventional catheters technologies, the location and force sensors generally utilize miniature wire-winded coils to which a ferrite core may be manually inserted with an open magnetic circuit configuration in order to yield sufficient induced-voltages suitable for location and force sensing of the catheter components. These conventional techniques are however time consuming and costly and involve delicate manual operations for fitting of the coils within the small dimensions of the catheters body.

However, other conventional techniques for coil fabrication are also less suited for fabrication of high induced voltage miniature open magnetic circuit coils, and are lacking, for instance with their ability to fabricate miniature open magnetic circuit coils with longitudinal dimension to lateral dimension aspect ratio of at least 3 or 4 (e.g., coil longitude of about 2 mm and lateral/width of about 0.6 mm). Accordingly, the conventional techniques are also lacking when it comes to fabrication of miniature open magnetic circuit coils having high sensitivity/induced-voltage (e.g., in the order of 0.1 to 1 μV/(Gauss*Hz)). This is because the sensitivity/induced-voltage typically depends on the longitudinal dimension to lateral dimension aspect ratio (where too small aspect ratio significantly decreases the effective permeability of the magnetic core as it depends on the geometry). For instance, conventional sheet-lamination technologies (also known as green sheet lamination), by which inductor coils are fabricated by printing internal inductor electrodes on a plurality of magnetic or dielectric green sheets and stacking the sheets, are not reliable and prone to manufacturing defects when it comes to fabrication of miniature coils with dimensions as described above and/or having sufficient length to width ratio needed to yield the high induced voltage.

Therefore, there is a need in the art for more efficient, cost-effective techniques for fabrication of such catheters and for the fabrication of small high sensitivity coils (i.e., having high induced voltage in response to magnetic fields) suitable for use for location and or force sensing in catheters such as those disclosed above. Moreover, there is a need in the art for automated fabrication techniques of such coils, suitable for mass production with yield of standardized coils having less variability between fabricated coils of the same design. Further, in the interest of further advent in the art of catheters, there is also a need in the art to facilitate even smaller and/or of higher impedance/induced-voltage, so as to improve either or both of the sensitivity and agility, and/or reduced dimensions, of the catheter.

Therefore, the present invention provides a novel positioning sensory system and novel configurations and fabrication methods for electric components including open magnetic circuit coil(s) suited for magnetic sensors of high induced-voltage, for example for use as position sensors to be furnished within narrow dimensions of a catheter body.

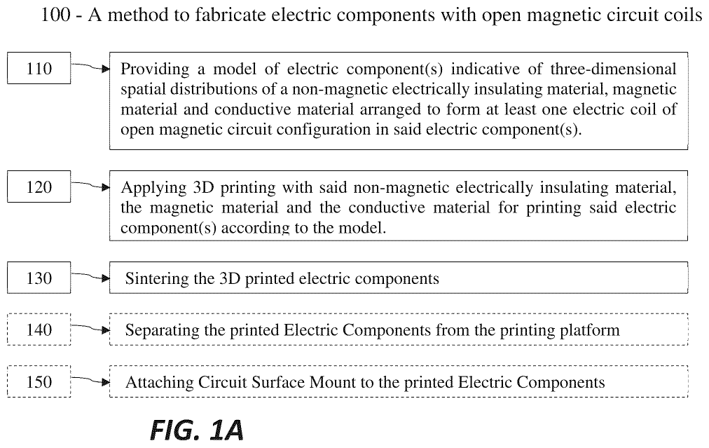

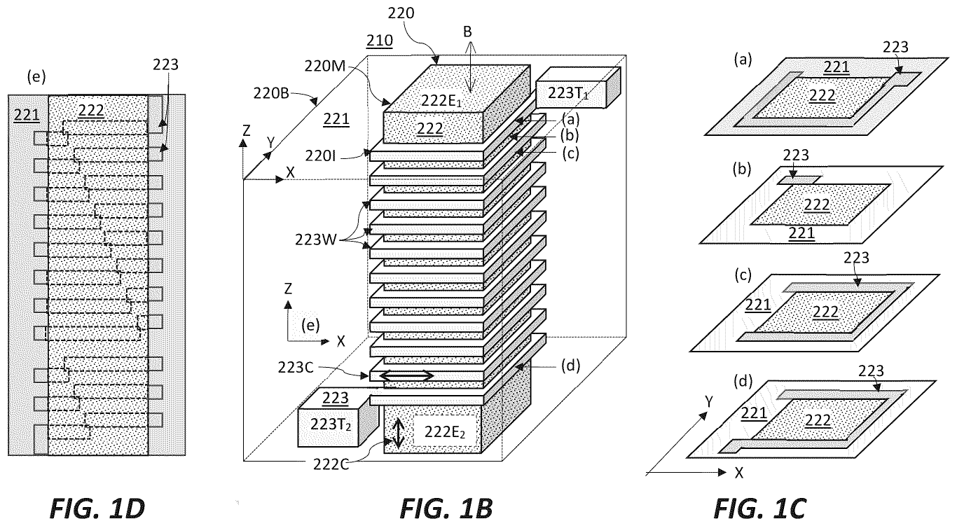

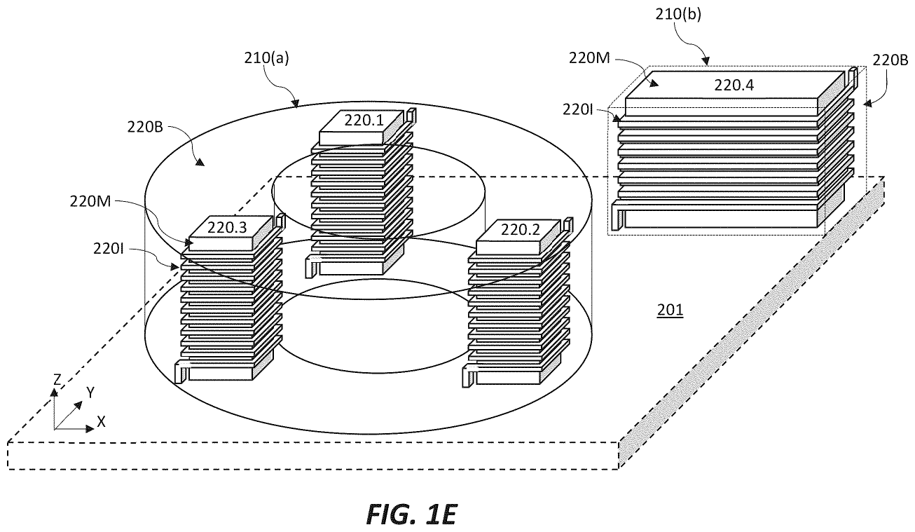

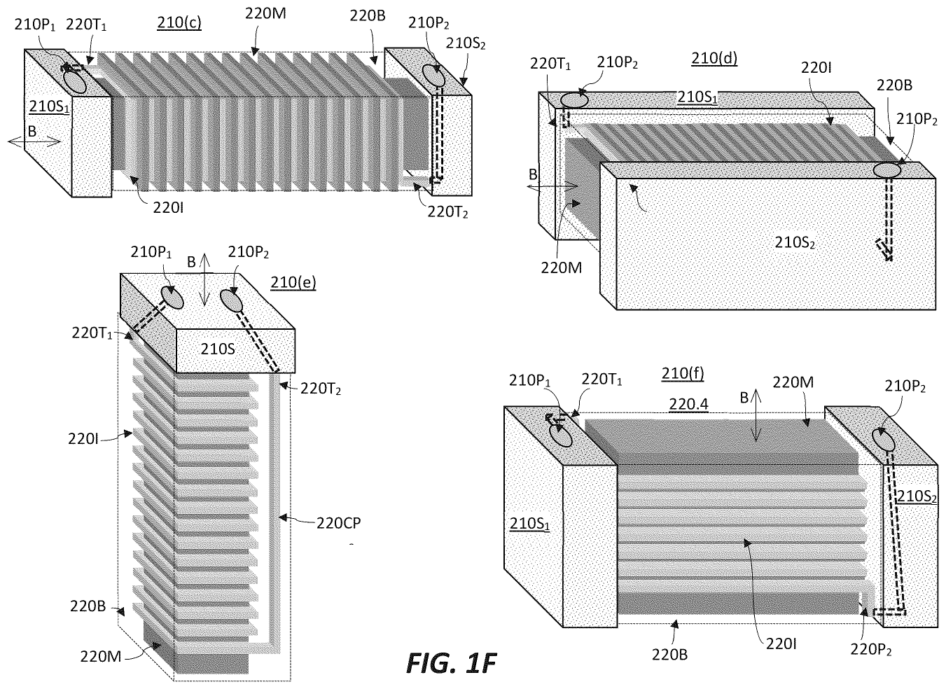

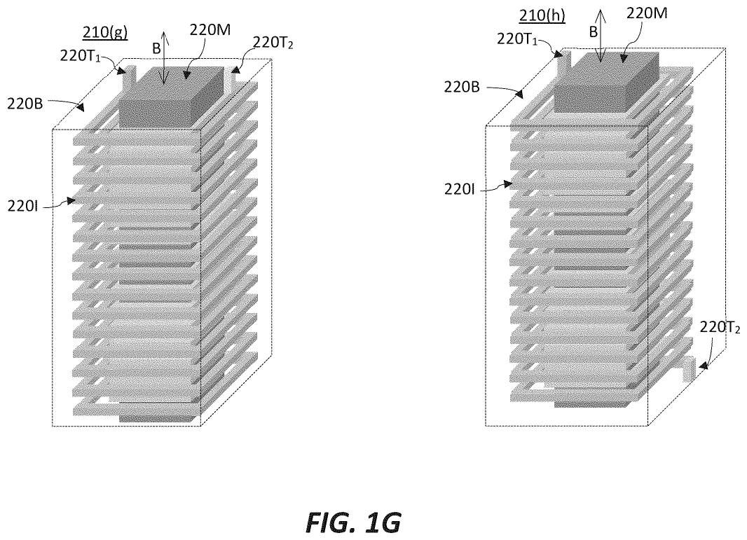

Reference is now made to FIG. 1A illustrating, in a flow diagram, a method 100 according to embodiments of the present invention, to fabricate electrical components with one or more electric coils therein having an open magnetic circuit configuration suitable for use in catheter's position sensor.

Advantageously, the method 100 utilizes a 3D-printing technology for printing electric components having ceramic and/or glass-ceramic solid main body (non-laminated bulk) with open magnetic circuit electric coils embedded therein. The open magnetic circuit electric coils embedded in the electric components are fabricated with at least three different materials which have different electromagnetic properties including: a magnetic material, a conductive material, and a non-magnetic dielectric (electrically insulating) material.

In this connection the phrase open magnetic circuit configuration in relation to the coils fabricated by the technique of the present invention should be understood herein as relating to coil structures that pose substantially no shielding of external magnetic fields (i.e., enabling the external magnetic field pass through the coil core without substantially bypassing around the core). The terms magnetic-material and/or particles is used herein to designate materials of high relative magnetic permeability μᵣ, for example having relative permeability preferably of μᵣ≥100 relative to the vacuum permeability μ₀. Conversely the terms non-magnetic-material and or particles are used herein to designate materials of low relative permeability μᵣ which is substantially lower than that of the magnetic materials used in the electronic components' fabrication and typically in the order of μᵣ˜1.

Moreover, it should be noted that the phrases multi-material three-dimensional-printing (i.e. here in after also referred to for short as 3D-printing) are used herein to designate multi-material 3D-printing techniques in which material adhesion, polymerization and/or curing are carried out layer by layer while enabling selective pixelated deposition, adhesion, polymerization and/or curing of voxels of several different materials (several distribution of different material voxels) in each layer. To this end, the 3D-printing (multi-material) technique (used in the present application should not be confused with other additive manufacturing techniques such as sheet-lamination techniques (e.g. green-sheet lamination), material extrusion or others, which do not facilitate selective pixelated deposition, adhesion, polymerization and/or curing of voxels of different materials within the same layer (a feature which is also referred to herein as in-layer pixelation).

Thus, in operation 110 of method 100, a model 200 of the one or more electric components 210 which are to be 3D-printed is provided. The model 200 is indicative 3D spatial distribution (voxel maps) of at least three materials, 221, 222, and 223, which are to be 3D-printed by multi-material 3D-printing in order to form the electrical components of the present invention. Particularly the model 200 is indicative of the respective distribution of the three materials 221, 222 and 223 according to which one or more open-magnetic circuit coils (typically of miniature dimension) are to be formed in the one or more electronic components 210 printed by the multi-material 3D-printing.

With reference to FIG. 1B a perspective view of a model 200 of one electric component 210 which includes in this non-limiting example a single open magnetic circuit coil 220 is schematically illustrated. As shown the model 200 is indicative of a 3D-distribution of conductive material 223 forming at least one electric channel 223C of the coil 220 extending between at least two end terminals 223T₁ and 223T₂ thereof which are typically formed by the same conductive material 223. Additionally, the model 220 is indicative of a 3D-distribution of magnetic material 222 forming at least one magnetic channel 222C extending between at least two distal-end facets thereof 222E₁ and 222E₂. Typically, the two distal-end facets 222E₁ and 222E₂ of the magnetic channel 222C present the input/output of magnetic flux flow through the magnetic channel 222C and accordingly the general direction/axis of sensitivity of the coil to external magnetic field B may typically be considered as the direction spanned between these distal-end facets 222E₁ and 222E₂, as shown by the double arrow of B in the figures (e.g. in some implementations regardless of the actual shape of the magnetic channel 3D printed between them).

The material distributions 222 and 223 forming the magnetic channel 222C and the electric channel 223C of the modeled coil 220, are configured in the model 220 with coiled geometry such that functionally an electric voltage would be produced in the electric channel 223C in response to a magnetic flux change in the magnetic channel 222C or vice-versa. The coiled geometry of the magnetic and electric channels, 222C and 223C, is generally such that at least one of the electric and magnetic channels, 223C or 222C, is configured to have a plurality of windings/turns (encircling) about the other one of the magnetic and electric channels, 222C or 223C. This thus provides that the electric channel is configured and operable as an inductor 220I of the electric coil 220 and the magnetic channel is configured and operable as a magnetic core 220M of the electric coil 220 to yield higher induced voltage in the coil 220 in response to magnetic fields changes and/or vice-versa.

As indicated above the electric component(s) 210 printed in the technique of the present invention includes at least one electric coil 220 having an open magnetic circuit configuration. The open magnetic circuit configuration facilitates efficient coupling of magnetic flux from an external magnetic field B to pass through the magnetic core 220M of the open magnetic circuit coil 220 thereby enabling yield of high induced voltage in response to magnetic flux changes. The open magnetic circuit configuration of the magnetic channel is typically implemented such that the distal-end facets 222E₁ and 222E₂ of the magnetic channel 222C are generally spaced apart (not connected to one another). Typically, the distal-end facets 222E₁ and 222E₂ are arranged to face different directions (e.g. opposite directions) so as to facilitate efficient flow of magnetic flux from an external magnetic field through the magnetic channel 222C. Moreover, the spatial distribution of magnetic material 222 in the magnetic channel 222C (between the distal-end facets 222E₁ and 222E₂ thereof) in the model 200 is such that the conductive channel 223C is not enclosed by a closed loop of the magnetic material 222. More specifically the spatial distribution of magnetic material 222 of the magnetic channel 222C does not define a continuous set of adjacent magnetic material voxels forming a closed path of the magnetic material surrounding the conductive channel 223C.

It should be noted that in the specific non-limiting examples shown in the figures the coiled geometry of the electric and magnetic channels 223C and 222C of the modeled coil 220 is implemented by a helical/spiral geometry of the electric channel 223C and with the magnetic channel 222C configured to occupy a region at the center of the helical/spiral geometry of the electric channel 223C. Thus, in these non-limiting examples the electric channel 223C is configured to have a plurality of windings/turns 223W about the magnetic channel 222C. Nonetheless, it should be appreciated that the present application is not limited by this specific geometry, and that models 200 presenting other coiled geometries of the magnetic and electric channels, 222C and 223C that functionally producing induction voltages/currents in the electric channel 223C in response to change in magnetic flux in the magnetic channel 222C or vice-versa may also be implemented/3D printed according to the technique of the invention without departing from the scope of the present Application.

In order to achieve the open magnetic circuit coil configuration effectively, the model 200 includes a spatial distribution of another material, being a non-magnetic electrically insulating material 221 to serve as a bulk material. The electric channel 223C between the two electric terminals 223T₁ and 223T₂ thereof, is fully embedded within the bulk material 221 such that voxels thereof interface either voxels of the bulk material 221, voxels of the magnetic material 222, or both, and are not located at edge facets of its respective electric components 210, except for the two electric terminals 223T₁ and 223T₂ which may optionally be exposed at edge facet(s) of the electric components 210 to facilitate electrical connection to the coil 220.

In this connection it should also be noted that the inductor 220I is preferably fully embedded within the bulk 220B of the electric component 220 such that the conductive material thereof 223 is not exposed at external surfaces of the bulk 220B (except possibly for the two electric terminals 223T₁ and 223T₂) and thereby isolated from environmental conditions which might degrade the physical/conductive properties of the conductive material 223 (e.g. due to excessive oxidation which might for example occur under the high temperature conditions of the sintering process; or storage in humid air). Specifically, it would be appreciated protection of the conductive material from environmental conditions is specifically important when implementing the technique of the present invention for the fabrication of miniature electric coil components. For example, the present invention facilitates fabrication of an open magnetic circuit coil 220 with conductive windings 223W having thickness as small 5μ along the electric channel 223C of the inductor 220I. Such small features may easily be damaged by environmental conditions, and therefore embedding them within the bulk 220B is particularly important in order preserve proper functionality of the coil, while allowing it to be subjected to some of the high temperature processes performed during the 3D printing without functional damage.

Turning back to FIG. 1A, in operation 120 of method 100, a multi-material 3D-printing is applied to print the one or more electric components 210 based on the model 200 to thereby obtain the one or more electric components 210 with the at least one open magnetic circuit electric coil 220 embedded therein. The multi-material 3D-printing may be carried out for example by utilizing a photopolymerization 3D-printing technique to print layers that are composed of a combination of several materials (e.g., Two, three or more) having different electromagnetic properties. For example, 3D printing of electric components with spatial distribution of at least three materials including: electrically-conductive material, magnetic material and non-magnetic dielectric material. With the technique of the invention electric components with small feature sizes can be printed utilizing 3D printed layers with thicknesses in the scale down to microns (e.g., sintered layer thickness of 8 microns) and with pixelated (in-later) features of characteristic sizes in the scale of tens of microns or less. The printing may be implemented for example based on the principles of Vat Photopolymerization, utilizing for instance stereolithography (SLA) or digital light processing (DLP) for the pattern implementation/projections. The invention is not limited to these specific technologies and may generally be implemented by other multi-material 3D printing technique as may be developed to be suitable for the fabrication of the electric components according to the invention.

For carrying out the 3D printing, curable resins corresponding respectively to the at least three materials designated by the model 200 are provided. More specifically the curable resins used in the 3D printing of the electronic components 210 according to the invention include at least the following:
A magnetic material resin which includes particles of a soft magnetic material magnetic material 222 suspended in curable polymer binder. The particles of the magnetic material 222 may include for example particles of Ceramic-Ferrite material. It should be noted that alternatively in some embodiments the particles of the magnetic material 222 may include particles of magnetic Metal Materials such as Iron alloy, iron-silicon alloy, iron-aluminum-silicon alloys, low-carbon steels, nickel-iron alloys, iron-cobalt alloys, ferrites, amorphous alloys, as well as in some cases Nickel-Cobalt Alloy (particularly in cases it has soft magnetic qualities). In embodiments where miniature coils are fabricated the particles of magnetic material used may be provided for example with characteristic sizes in the micron to submicron scale in embodiments where the magnetic channel 220M of the coil manifests delicate features.
A conductive material resin which includes particles of conductive material 223 suspended in curable polymer binder. The particles of the conductive material 223 may for instance include Silver material, Copper material and/or Silver-Palladium alloy. As specific choice of suitable conductive material may generally depend on the required temperatures of the sintering/firing process required to finalize the printing of the electric components and may thus depend on the types of magnetic and dielectric materials. In embodiments where miniature coils are fabricated having conductive windings with characteristic sizes in the scale of several microns or few tens of microns, the particles of conductive material 223 used in the printing may have characteristic sizes in the submicron scale and as small as in the nanometer scale in cases where the printing of such small features is desired.
A dielectric and non-magnetic material resin which includes particles of non-magnetic dielectric material 221 suspended in curable polymer binder. The particles of the non-magnetic dielectric material typically include ceramic or glass-ceramic particles which can withstand the high sintering/firing temperatures required for sintering the printed conductive and magnetic materials. Also, here the size of the particles may be selected according to the minimal feature sizes that should be occupied by the dielectric material/bulk of the electric components, and for fabrication of miniature coils may be selected for instance in the order of nanometer to submicron scale.

In regards to the material selection in the above, it should be noted that although printing magnetic channel 222C with magnetic metal material is generally possible, in various embodiments printing the magnetic channel with an electrically insulating ceramic ferrite may be preferred. One reason for that is that the use ceramic ferrite facilitates achieving more compact arrangement of magnetic and electric channels, 222C and 223C. This is because soft magnetic metal materials (non-ceramic ferrite) that are suitable for use for the magnetic channel printing, are typically conductive. Therefore, a magnetic channel 222C printed with the non-ceramic ferrite should be spaced from the electric channel 223C (in order to prevent short circuit). This for one requires design and printing of a more complex model 200 of the electric component in which intermediate voxels of the dielectric materials (buffer of ceramic or glass-ceramic material) should be located between the magnetic channel 222C and the electric channel 223C at regions where these channels may otherwise interface one another. Additionally, it may impact the achievable miniaturization of the component due to the required separation between the conductive magnetic channel 222C and the electric channel 223C that should preferably be of at least a few microns for sufficient electric isolation. Moreover, when using such non-ceramic ferrite materials, and with present printing equipment, the separation is in practice larger typically in the order of 10 (s) or more microns, according to the minimal lateral (in-layer) characteristic sizes supported by present printing equipment. Therefore preferably, ceramic ferrite material is used for the printing of the magnetic channel 222C, and particularly when printing of electric components including miniature coils having high sensitivity/induced-voltages.

Thus, utilizing the at least three curable raisings described above, 3D printing of the electric components 210 may be performed layer by layer according to the three-dimensional spatial distribution of the materials in the model 200.

As indicated above the printing may be for example be carried out utilizing photopolymerization multilateral 3D printing techniques such as SLA or DLP. Preferably the printing direction by which the printing is conducted is substantially perpendicular to planes spanned by the conductive windings 223W. I.e., the printing directions is preferably the Z direction illustrated in FIG. 1B which is perpendicular to the X-Y planes about which the windings are (approximately) spanned. With the current 3D-printing technologies, this choice of the printing direction Z may have particular importance in the printing of miniature open magnetic circuit coils 200, in order to achieve a high-density of windings in such miniature coils (e.g., high count of conductive windings 223W of the inductor 220I per unit length of the magnetic core 220M). This is because with the current 3D-printing technologies the lateral feature resolution in the X-Y plane is lower than the “vertical” resolution obtained along the printing direction z of the layers (e.g., the lateral resolution of some 3d printing technologies facilitates small feature sizes of about 25 μm and even down to 15 μm while the thicknesses of the printed layers (and accordingly the “vertical” feature sizes), may be substantially smaller, below 10 μm and even down to only one or few microns (e.g. 5 μm).

With reference to FIGS. 1C and 1D, four successive lateral cross-sections (a), (b), (c) and (d) of a model 200 of an electric coil 220 according to an example of the present invention are schematically illustrated in self-explanatory manner in FIG. 1C, as well as a vertical cross-section (e) which is schematically illustrated in self-explanatory manner in FIG. 1D. The lateral cross-sections (a), (b), (c) and (d) are taken from lateral planes X-Y perpendicular to the printing direction Z and present four respective printable layers of the model 200. In an example the cross-sections (a), (b), (c) illustrate three consecutive layers of the electric coil 220.

To this end, typically the 3D printing of each layer of the open magnetic circuit electric coil(s) 220 typically includes the following (not necessarily in that order):
printing and curing the magnetic material 222 at regions of the layer corresponding to the magnetic core(s) 220M of the electric coils;
printing and curing the conductive material 223 at regions of the layer corresponding to inductor(s) 220I of the electric coil(s); and
printing and curing the non-electrically-conductive (dielectric) non-magnetic material 221 at least near interfaces of the conductive material 223 (possibly excluding the contact regions 223T₁ and 223T₂) which are not at interface with the magnetic material 222 (this is in order to embed the conductive material 223 in the bulk of the electric component 210 for protection against degradation due to environmental conditions.

Thus, by the 3D printing 120, the structures of one or more electric component(s) 210 which include at least one electric coil 220 of open magnetic circuit configuration is obtained.

In the present example, as shown by FIGS. 1B to 1D, the 3D printing is carried out such that the magnetic core 220M of the open magnetic circuit coil 220 of the occupies the central region of the coil/helical geometry of inductor 220I while not enclosing the conductive windings of the inductor with a continuous closed loop/channel of the magnetic material 222.

In this connection, it should be noted that in some embodiments of the present invention the method 100 is carried out for 3D-printing electric components 110 in which miniature electric coils having features of micron scale size are embedded. For instance, the 3D printed layers may be printed with thicknesses in a range of about 5 to 15 μm (e.g., 9 μm), depending on the specific model design and 3D-Printing technology used. Accordingly, a pitch of the coiled/helical arrangement of windings 223W, which may be in the order of twice the pitch of the 3D printed layers, may be within the range of about 10 μm to 30 μm along the printing direction Z of the 3D printed layers (e.g., winding pitch of about 18 μm).

It should be understood that although in the above described non limiting examples the coil windings 223W are distributed at different 3D printed layers of the electric component 210 and have helical configuration, other winding configurations may be implemented in the open magnetic circuit electric coils that are fabricated by the technique of the present invention without departing from the scope of the present Application. For instance, a plurality of windings may be arranged in each of one or more of the 3D printed layers of the electric component 210 and may be connected in a spiral geometry within such layers or in form of a concentric double-/multi-helix configuration across multiple layers as illustrated for instance in FIG. 1G. Accordingly open magnetic circuit coils with windings 223W of a single-helix geometry may be fabricated and/or in order to facilitate higher density of the windings 223W within a miniature coil, the windings 223W may be configured and fabricated by the technique of the invention with a concentric multi-helix geometry and/or with a spiral-helical geometry. For instance, in a spiral-helical arrangement of windings, a one or more spiral windings may be distributed at different one or more 3D printed layers within the 220B bulk and connected between them to form the spiral-helical arrangement. Alternatively, or additionally, in a double-/multi-helix geometries of the windings, concentric helixes (or spiral-helixes) of windings which may be connected in parallel or series to one another, may be fabricated within the open magnetic circuit coil 220.

In such embodiments where concentric windings 223W or parts thereof are fabricated within the same 3D printed layer, the concentric parts are typically spaced by the 3D printed non-magnetic dielectric material 221 in order to preserve the open magnetic circuit geometry of the coil 220 while also not shortcutting the windings' path. In some embodiments the 3D printing is performed with lateral printing resolution of the 3D printed layers in the order of 15 to 30 μm (typically about 25 μm). In this regard it would be appreciated that the projector typically projects pixel with resolution between 19 μm to 35 μm and during the sintering of the printed components their dimensions shrink by about 20% thus yielding in layer resolution/feature-sizes between 15 to 30 μm.

Accordingly, in some implementations of the invention where miniature coil 220 is fabricated with inclusion of concentric in-layer windings 223W (e.g., with multi-helix or spiral-helix geometries), a lateral pitch of in-layer windings may be in the order of about 30 μm to 60 μm (e.g., typically about 50 μm), being about twice the lateral resolution of the 3D printing. This may thereby be implemented to further improve the spatial densities of the windings in the miniature coils and yield improvement in the voltage induced in response to varying magnetic fields.

With reference to FIG. 1E, two examples of electric components 210(a) and 210(b) are shown on a printing platform 201, after being printed by operations 110 and 120 of the method 100 of the invention. The two electric components exemplified here are made suitable for use as sensors for sensing the location and/or orientation of medical instrument such as a catheter (e.g., relative to an external frame of reference)—component 210(b), and for sensing the force applied by the medical instrument/catheter to a tissue in contact therewith—component 210(a). In this particular nonlimiting example the electric component 210(a) has a ceramic or glass-ceramic bulk 220B formed with a ring/donut shape with three miniature open magnetic circuit coils 220.1, 220.2 and 220.3 of substantially similar properties distributed over the circumference of the ring/donut shapes bulk 220B and co-aligned with their magnetic axes along a symmetry axis Z of the donut shape's bulk. Accordingly, this electric component may be fitted within a catheter's elongated body with its symmetry axis Z aligned to the longitudinal axis of the catheter's body and is suitable for sensing its orientation relative to a magnetic field source placed in front of it along the longitudinal axis at a distal part of the catheter (thus may serve for sensing a force applied by the catheter to a tissue). In this particular nonlimiting example, the component 210(b) is a single open magnetic circuit coil with small height along the Z direction of its magnetic axis and having elongated lateral aspect ratio over the X-Y plane, making it suitable for fitting within a catheter's elongated body for sensing a component of a magnetic field perpendicular to the longitudinal axis Z of the catheters elongated body with sufficient sensitivity. Accordingly, two such components 210(b) arranged in the catheter with different directions of their magnetic axes perpendicular to the catheters longitude, together with one of the coils of the component 210(a) whose magnetic axis is aligned with the catheters longitude, may be used to sense the position and orientation of the catheter relative to a source of external magnetic field residing externally from the catheter.

Turning back to FIG. 1A, method 100 further includes operation 130 for sintering the one or more 3D structures forming the electric components 210 printed in the 3D-printing operation 120. The sintering is generally performed at temperature sufficient for burning or driving off polymers (binding polymers) from the printed electric components 210 as well as firing ceramic or glass ceramic materials used in the printing. To this end, after the sintering the material density of the printed ceramic and metal materials (e.g., 221, 222 and 223) of the electric components 210 approaches 100%. For example, in embodiments where the conductive material 223 mainly includes Silver, the sintering may be performed at temperatures below the silver melting point (e.g., at temperatures not exceeding 961° C. (preferable in this case the sintering temperature may be limited up to about 900 C in order to achieve a stable sintering process). In other embodiments where the conductive material includes a Silver-Palladium alloy, the sintering may be performed at temperatures below the alloy melting point (the specific melting point depends on the ratio of silver and palladium in the alloy and depending on the composition the melting point is generally between the melting point of silver, 961° C., and that of palladium, 1,555° C.). In another example, in embodiments where the conductive material 223 includes Copper, the sintering may be performed in an Oxygen deprived environment (so as to avoid oxidation of the Copper), and at temperatures below the 1084° C. Copper melting point.

Further, optionally (depending on the specific printing technology used), the method 100 may include operation 140 for separating the electric components 210 from the building platform (e.g., 201 shown in FIG. 1E). Generally, depending on the type of building platform 201 (e.g., and whether it can withstand the sintering 130), the operation 140 is typically performed before the sintering 130. Indeed, in some embodiments of the invention, particularly where miniature/delicate electric components are printed, which may be damaged by carrying out their separation from the building platform before the sintering, a suitable building 201 platform may be used which can withstand the temperatures of the sintering 130, and the components separation from the platform may be conducted post sintering so as to avoid/reduce damage to the components during the separation process.

Further, optionally, in some embodiments it is also desired to furnish one or more of the 3D printed electric components 210 with surface mounts for their installation on a circuit board via surface mount technology (SMT). According to some embodiments of the invention, surface mounts suitable for the SMT are 3D-printed together with the 3D-printing of the components 210. In such embodiments typically materials such as silver-palladium alloy are used for the SMT mounts' electric contacts, since the conventional materials, Tin and Nickel-Tin which are typically used in SMT mounts' electric contacts, are not suitable for the sintering process of the 3D printing technology of the invention (Tin's melting point is too low and Nickel-Tin will oxidize in the process). To this end in some embodiments the 3D-printing operation 120 of method 100 further includes 3D printing of the external/SMT contacts. The 3D printed SMT contacts may be for example fabricated from silver-palladium alloy (Ag—Pd) and formed with thickness of about 9 μm to yield external Ag—Pd SMT electrodes for the 3D printed electric components 210.

Alternatively, or additionally, in some embodiments of the invention the SMT contacts/mounts, or some of them, are fabricated/attached to the 3D printed electric components 210 only after the 3D-printing and Sintering of the components 210. To this end, in some embodiments method 100 further includes operation 150 for fabricating/attaching SMT surface mounts to the 3D printed electric components 210. With reference to FIG. 1F, four electric components 210(c), 210(d), 210(e) and 210(f) each including a single open magnetic circuit coil 3D-printed by the technique of the present invention are illustrated in self-explanatory manner. The four electric components 210(c), 210(d), 210(e) and 210(f), are shown with different variations of surface mounts 210S, 210S₁ and 210S₂ coupled and electrically connected to the 3D-printed components. In this example, the surface mounts 210S, 210S₁ and 210S₂ arranged at each coil components, include a pair of electric contacts/pads (e.g., made of Tin or Nickel+Tin) 210P₁ and 210P₂ which are electrically connected to the respective electric terminals 220T₁ and 220TP₂ of the respective coils 220 of the components and are suitable for soldering to a circuit board via SMD technology. As shown in the figure, in the two variations of the electric components 210(c) and 210(d), the SMT mounts are furnished such that the coils 220 will be mounted to a circuit board with a so-called horizontal orientation, such that their magnetic axis B is parallel to a surface of the circuit board. The components 210(c) and 210(d) differ by the respective arrangement of the SMT mounts where in 210(c) the mounts are arranged along the magnetic axis B (to yield a more narrow and elongated dimensions of the component), and in 210(d) the mounts are arranged aside/parallel to the magnetic axis B are 210(d) (to yield shorter and wider dimensions of the component). In the two variations of the electric components 210(e) and 210(f), the SMT mounts are furnished such that the coils 220 will be mounted to a circuit board with a so-called vertical orientation, such that their magnetic axis B is perpendicular to a surface of the circuit board. The components 210(e) and 210(f) differ by the respective arrangement of the SMT mounts where in 210(e) a single mount is arranged along the magnetic axis B (to yield a narrower and more elongated/taller dimensions of the component), and in 210(f) the mounts are arranged aside/parallel to the magnetic axis B are 210(d) (to yield shorted and wider dimensions of the component). Accordingly, as shown in the figure the present invention facilitates different arrangements of the SMT mounts on the 3D-printed electric components to yield desired optimization of the total dimensions of the components with the mounts to fit desired real-estate space allocated thereto inside a device, such as a catheter in which there are installed, as well as facilitating proper selection of the magnetic axis orientation of the components relative to the circuit board—to facilitate their proper accommodation for sensory application in which the available real-estate for their placement is in shortage. Notably, it should be understood that the fabrication of the SMT mounts 210S, 210S₁ and 210S₂ may be carried out according to any suitable technique known in the art (e.g., by electroless or electrochemical deposition of for example Nickel (Ni) and/or Tin (Sn)), or during the 3D-Printing in 120 as indicated above. Also notably, that in embodiments where the SMT mounts 210S, 210S₁ and 210S₂ are arranged to face the magnetic axis/es of the coil(s), their main body is preferably made of non-magnetically-permeable material so as not to screen magnetic flux from the coil.

With reference together to FIGS. 1E to 1G, several variations of electric components 210(a) to 210(g) with 3D-printed open magnetic coils according to the present invention are illustrated. As shown in the coils of all of those electric components 210(a) to 210(g), the boundaries of the bulk 220B of the electric components/coils are printed with one or more materials other than the conductive material 223 so as to protect the conductive material of the inductor 220I from damage due to environmental conditions. In other words, the conductive material of the inductor 220I is fully embedded within a bulk 220B of its respective electronic component and not exposed at external surfaces of the bulk except 220B from electric contacts thereof. As indicated above, spacings between adjacent conductive windings may be occupied by 3D printed non-electrically-conductive material (e.g., the dielectric bulk material 221, or the magnetic material 222 in case it is not conductive). For example, as shown for instance in components 210(c) and 210(d) the conductive windings of the inductor 220I may be spaced from one another by the magnetic material 223 of the magnetic core 220M, (which is an insulator in these examples). This is as long as the magnetic material 223 does not form a closed loop over the conductive windings, and thus preserve the open magnetic circuit configuration of the coil. Moreover, in some embodiments the magnetic material may extend in places to the boundary of the electric component, as shown for instance in the component 210(c). In other embodiments, e.g. component 210(e), the windings may be spaced by the dielectric bulk material 221. As shown in the component 210(e), in some embodiments the model of the electric component may also include a model for extending a conductive path 220CP from the inductor 220I of the coil to a terminal electric contact thereof, e.g., 220T₂ or to another component located any place in the bulk 220B. Furthermore, as indicated above, the inductor 220I may be fabricated as with a coaxial/multi-helical configuration, as shown for example in the double helix of the component 210(g), and/or with spiral-helical configuration as shown for example in the component 210(h).

To this end the technique of the present invention provides miniature electric components having a ceramic/glass-ceramic body embedding therein highly sensitive open magnetic circuit coils embedded which may be configured and operable as sensors (e.g., magnetic sensors and/or position sensors and/or orientation sensors and/or force sensors). The miniature electric components may be fabricated with small dimensions, for example in the order of ˜0.5 mm to ˜2 mm and with high sensitivity/induced-voltage in response to magnetic fields, for example in the range ˜0.1 to 1 μV/(Gauss*Hz).

It should be noted that the illustrations of the electrical components in FIGS. 1B to 1G, are provided for illustrative purposes only, and are not necessarily drawn to scale or with proper proportions. For example, generally the count of winding in open circuit electric coils 220 according to the present invention may be substantially higher than what is illustrated in the figure. Also, open circuit electric coils 220 are illustrated with rectangular shapes/cross-sections, whereby it should be understood that in practice they may be 3D-printed with various polygonal or rounded shapes/cross-sections, and or with different aspect ratios than those illustrated, without departing from the scope of the present invention.

In view of the above, method 100 of the present invention provides a novel and inventive technique for fabrication of electric components including open magnetic circuit coils via 3D printings. The method 100 is suited for fabrication of miniature coils with features sizes as small as few microns, and may be used in mass production (particularly when the printing is based on vat photopolymerization technology), in order to simultaneously 3D print large pluralities (e.g., typically between hundreds and several thousand electric components built in parallel on the same platform) of miniature coils in one printing batch. This makes the technique of the present invention cost effective for printing miniature coils which typically otherwise require one-by-one fabrication while typically requiring manual fabrication steps, when fabricated by the conventional techniques.

To this end the present invention also provides novel and inventive electric component formed by 3D printing of at least three different 3D-printed materials and including one or more electric coils of open circuit configuration. The electric components as exemplified for instance by FIGS. 1E to 1G include a bulk formed by non-magnetic and electrically insulating 3D printed material with at least one electric coil in the bulk including: (a) at least one magnetic channel 222C formed by 3D-printed magnetic material embedded in the bulk; and (b) at least one electric channel 223C formed by 3D-printed conductive material embedded in the bulk and extending therethrough between two terminals which may be exposed at a surface of the bulk to serve as electric contacts for the electric coil. At least one of the electric or magnetic channels is 3D printed with coiled geometry to form a plurality of windings about the other one such that the electric channel functions as an inductor of the coil and the magnetic channel as a magnetic core thereof (for example the electric channel may be 3D-printed with a coil/helical geometry having a plurality of windings about the magnetic channel). The 3D printed magnetic channel which forms the magnetic core is configured as an open magnetic circuit configuration such that it does not enclose the conductive channel with a closed loop of magnetic material.

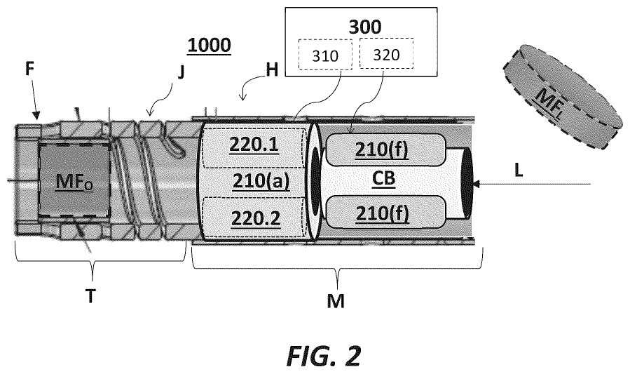

As indicated above the 3D printed electric components 210 of the present invention with the open magnetic circuit coils 220 therein are particularly suited for use as position and/or force sensors in medical instrument requiring the same. Reference is made to FIG. 2 illustrating a side-view of a medical instrument 1000 (e.g., probe/catheter) according to an embodiment of the present invention in which position and force sensors utilizing the 3D printed electric components 210 of the present invention with the open magnetic sensor coils therein for sensing the spatial location and orientation of the medical instrument as well as the force applied thereby to a tissue in contact therewith. The medical instrument 1000 includes a housing H with a magnetic field-based position sensor 300 furnished therein. The medical instrument 1000 is associated with one or more magnetic field sources MF_{L} and/or MF_{O} which provide reference magnetic fields which can be measured by the magnetic field-based position sensor 300 to respectively determine the spatial location and orientation of the medical instrument 1000 and/or the force applied thereby to a patient's tissue.

In an embodiment where force applied by the medical instrument 1000 to a body tissue, the medical instrument's housing H may be arranged along longitudinal axis L and include a main section M and a tip section T coupled at a distal end of the main section M via a banding coupler J. The banding coupler J, which may be for example a spring or joint, is configured such that the orientation of the tip section T can be bent relative to the longitudinal axis L of the housing H under applied force F (which may be applied for example when tip section T of the medical instrument is in contact with a body tissue of a patient) with banding degree corresponding to a magnitude and direction of the applied force F. In order to measure the magnitude and direction of a force F applied by the tip T to a tissue, the medical instrument 1000 includes a first magnetic field source MF_{O} (e.g., magnetic field generating coil with associated power delivery circuitry) and a magnetic-field-based positioning sensor 300. The orientation sensor 310 and the first magnetic field source MF_{O} are arranged in the housing at different sides of the joint J, for instance typically the first magnetic field source MF_{O} is arranged in the tip section T and the orientation sensor 310 at the main section M (although opposite arrangement may also be applicable). The first magnetic field source MF_{O} is configured and arranged in the housing H (e.g. in the tip T) for producing a magnetic field with flux lines/axis generally along the longitudinal axis L of the housing H. The orientation sensor 310 includes a plurality of at least three open magnetic circuit coils 220.1, 220.2 and 220.3 according to the present invention which are capable of sensing the magnetic field generated by the first magnetic field source MF_{O}. The three open magnetic circuit coils 220.1, 220.2 and 220.3 of the orientation sensor 310 are generally arranged with their magnetic axes collinear to one another and parallel to the longitudinal axis L of the housing H while not being co-planar, so that a relative orientation between the orientation sensor 310 and the magnetic field source MF_{O} can be determined about two orientation axes (e.g. pitch and yaw) by a magnitude of the magnetic field for the source MF_{O} sensed by the three coils, as will be appreciated by those versed in the art. In the present nonlimiting example, the housing has a narrow width/diameter of about 2 mm, and the three open magnetic circuit coils 220.1, 220.2 and 220.3 are parts of the electric component 210(a) illustrated in FIG. 1E, which is fabricated according to the technique of the present invention. As indicated above, the electric component 210(a) which has solid ceramic or glass-ceramic bulk/body is fitted in the main section M of the housing and optionally connected directly to signal lines (not specifically shown) for receiving the coils signals without a need for an intermediate circuit board (the signal lines may be soldered directly to the terminals of the coils 220.1, 220.2 and 220.3 in the electric component 210(a)). Preferably the ceramic or glass-ceramic body of the electric component 210(a) has a cylindrical/donut structure as shown above with a hole in the middle and is fitted with its symmetry axis parallel to the longitudinal axis of the housing such that various parts/connection lines of the instrument can be arranged to pass therethrough from the main section of the housing to the tip section (for instance passage of power delivery lines to the magnetic field source and/or other connection/fluid lines which may be furnished in the medical instrument). Advantageously, utilizing the configuration of the electric component 210(a) for the orientation sensor 310 facilitates reliable, accurate and cost-effective fitting and electrical connection of the three open magnetic circuit coils 220.1, 220.2 and 220.3 within the narrow dimensions of the housing H. It should be noted that in alternative embodiment the orientation sensor 310 may include the three open magnetic circuit coils 220.1, 220.2 and 220.3 as separate electric components fabricated according to the present invention. For instance, the orientation sensor 310 may include a rigid flat circuit board (e.g., disk like shaped having a hole in the middle) with three electronic component such as 210(e) described above with reference to FIG. 1F furnished vertically on the rigid rounded circuit board by their SMT contacts such that the open magnetic circuit coils embedded therein serve as the coils 220.1, 220.2 and 220.3 the orientation sensor 310. In yet another embodiment the orientation sensor 310 may be implemented with a flexible/foil circuit board, such as the flex board CB of the location sensor 320 discussed below, which is flexed about the longitudinal axis L of the housing H to a cylindrical shape. Three electronic component such as 210(c) described above with reference to FIG. 1F may be furnished horizontally on cylindrical flexed board (e.g., CB) by their SMT contacts such that their magnetic axes are parallel to the longitudinal axis L thus providing that the open magnetic circuit coils embedded therein serve as the coils 220.1, 220.2 and 220.3 the orientation sensor 310. To this end, having considered the technique of the present invention person of ordinary skill in the art will readily appreciate how the orientation of the sensor 310 relative to the magnetic field source MF_{O} may be determined based on measurements of the magnetic field generated by the source MF_{O}. Moreover, a person of ordinary skill in the art will readily appreciate how the force vector F applied to the tissue may be determined based on the measured orientation and the properties (e.g., spring constant) of the banding coupler J.

Alternatively, or additionally, in some embodiments the position sensor 300 includes a location & orientation sensor 320 that is adapted to measure the spatial location and orientation of the medical instrument 1000 relative to a frame of reference provided by magnetic fields from at least one external magnetic field source MF_{L} (located externally to the medical instrument). The location & orientation sensor 320 includes three open magnetic circuit coils 220 configured according to the technique of the present invention and arranged in the housing H such that their magnetic axes are not parallel to one another and span three dimensional coordinates. Accordingly, the three open magnetic circuit coils 220 facilitate measurement of different vector components of the magnetic field generated by the external magnetic field source MF_{L}, and by such measurement, as will be readily be appreciated by those versed in the art, a location and orientation of the medical instrument 1000 can be determined relative to the reference frame defined by the magnetic field of the external magnetic field source MF_{L}.

In embodiments, as for instance illustrated in the figure, the location & orientation sensor 320 includes a flexible circuit board CB folded to a cylindrical shape, with at least two preferably similar open magnetic circuit coils 220 according to the present invention, furnished on the flexible circuit board CB such that their magnetic axes are substantially not parallel to one another and also not parallel (typically perpendicular) to the longitudinal axis L of the housing H. The two preferably similar open magnetic circuit coils 220 may be for example coils implemented by electronic components such as 210(b) of the present invention which have high magnetic sensitivity while with low profile/height along their magnetic axes so that they can be fitted within the narrow housing with their magnetic axes perpendicular to the longitudinal axis L of the housing H. Preferably, as shown in the figure, the two open magnetic circuit coils 220 may be implemented by electronic components such as 210(f) of the present invention, which are similar to 210(b) only having SMT contacts fitted thereon. Accordingly, the electronic components 210(f) with the two coils thereof can be furnished on the flexible board CB by cost effective SMT mounting technology. In addition, the location & orientation sensor 320 includes a third open magnetic circuit coil arranged in the housing such that its magnetic axis is not parallel to the magnetic axes of the two other coils, and preferably such that its magnetic axis is substantially parallel to the longitudinal axis L of the housing H. In embodiments as illustrated in the figure, where the position sensor 300 includes also the orientation sensor 310, the third open magnetic circuit coil of the location & orientation sensor 320 may be one of the coils of the orientation sensor 310, for example coil 220.1. Alternatively, for instance in embodiments where the position sensor 300 does not include an orientation sensor 310, the third open magnetic circuit coil may also be arranged on the flexible board CB (e.g., with its axis substantially parallel to the longitudinal axis L). In such embodiments the third coil may be implemented by an electric component of the invention, for example by electric component 210(c) illustrated in FIG. 1F, and may optionally be furnished on the flexible board CB via SMT technology.

Thus, the present invention provides novel and inventive position sensor 300 configuration, utilizing novel and inventive 3D-printed open magnetic circuit coils to facilitate cost effective and reliable fitting of the position within medical instruments of narrow dimensions. As indicated above the position sensor 300 may include an orientation sensor 310 suitable for example for determining a force applied by the medical instrument to a tissue and including three open magnetic circuit coils configured according to the present invention, and/or a location & orientation sensor 320 including three open magnetic circuit coils configured according to the present invention and operable to determine the spatial location and orientation of the medical instrument 1000 (in cases where the position sensor 300 includes both sensors 310 and 320, a total of five open magnetic circuit coils may be sufficient for carrying out the measurements).

### Examples

Example 1. An electric component comprising one or more electric coils, the electric component is formed by 3D printing of at least three different 3D-printed materials with spatial distributions yielding an open magnetic circuit configuration of at least one electric coil of the one or more electric coils; wherein the electric component include:
a bulk formed by non-magnetic and dielectric 3D printed material; and
at least one electric coil of the open magnetic circuit configuration, 3D-printed in the bulk, including:
   at least one magnetic channel comprising magnetic material 3D-printed in the bulk forming a magnetic core of the at least one electric coil; and
   at least one electric channel forming an inductor of the at least one electric coil comprising conductive material 3D-printed in the bulk with a coil/helical geometry having a plurality of electrically connected conductive windings arranged to circumference the magnetic channel of the magnetic core; and
   wherein the magnetic channel of the magnetic core is configured and operable with the open magnetic circuit configuration and includes magnetic material occupying a central region of the coil/helical geometry of the inductor while not enclosing the conductive windings with a closed loop of the magnetic material.

Example 2. The electric component according to Example 1 wherein the electrically connected conductive windings include conductive windings distributed at different 3D printed layers of the electric component thereby forming a helical arrangement of electrically connected conductive windings.

Example 3. The electric component according to Example 2 wherein a thickness of the 3D printed layers is in the order of 5 to 15 μm thereby yielding a pitch of the helical arrangement of conductive windings in the order of twice the pitch of the 3D printed layers being about 10 μm to 30 μm along a printing direction of the 3D printed layers.

Example 4. The electric component according to Example 1 wherein the electrically connected conductive windings include plurality of conductive windings arranged concentrically in one or more 3D printed layers of the electric component thereby forming a spiral arrangement of conductive windings in the one or more 3D printed layers.

Example 5. The electric component according to Example 4 wherein a resolution of the 3D printed layers, after the sintering, is in the order of 15 to 30 μm, thereby yielding a pitch of the spiral arrangement of conductive windings in the order of twice the resolution of the 3D printed layers with respect to a lateral plane of the 3D printed layers.

Example 6. The electric component according to Example 5 wherein the electrically connected conductive windings include a plurality of the spiral arrangement of conductive windings distributed at different 3D printed layers of the bulk and electrically connected between them to form a spiral-helical arrangement of the electrically connected conductive windings.

Example 7. The electric component according to Example 1, wherein spacings between adjacent windings of the plurality of electrically connected conductive windings are occupied by 3D printed non-electrically-conductive material being one of the non-magnetic dielectric material and the magnetic material.

Example 8. The electric component according to Example 1, wherein the 3D-printed conductive material of the inductor is fully embedded within the bulk and not exposed at external surfaces of the bulk, so as to isolate the inductor from environmental conditions which might degrade its physical/conductive properties.

Example 9. The electric component according to Example 1 wherein at least one of the following:
the conductive material includes Silver;
the conductive material includes Copper;
the conductive material includes Silver-Palladium alloy;
the magnetic material includes Ceramic-Ferrite material;
the magnetic material includes Metal Material;
the magnetic material is a soft magnetic material having relative permeability μᵣ in the order of 100 or more
the non-magnetic dielectric material includes ceramic or glass-ceramic material.

Example 10. A method to fabricate one or more electric components including one or more electric coils;
wherein at least one electric coil of the one or more electric coils has an open magnetic circuit configuration;
the method includes:
providing a model of the one or more electric components wherein the model is indicative of three-dimensional spatial distribution of at least three materials in the at least one electric coil having the open magnetic circuit configuration, including: 3D-distribution of magnetic material, 3D-distribution of conductive material, and 3D-distribution of non-magnetic dielectric material;
wherein the three-dimensional spatial distribution of the at least three materials is indicative of:
   spatial distribution of a bulk of the at least one electric coil formed by non-magnetic dielectric material;
   spatial distribution of magnetic material, defining at least one magnetic channel in the bulk associated with a magnetic core of the at least one electric coil;
   spatial distribution of conductive material defining at least one electric channel in the bulk associated with an inductor of the at least one electric coil and having a coiled/helical geometry with a plurality of electrically connected conductive windings arranged to circumference the magnetic channel;
   wherein the spatial distribution of the magnetic material has the open magnetic circuit configuration and is indicative of magnetic material occupying a central region of the coil/helical geometry of the inductor while not enclosing the conductive windings of the inductor with a closed loop of the magnetic material; and
   applying 3D printing to print the one or more electric components thereby obtaining the at least one electric coil with the open magnetic circuit configuration facilitating efficient coupling of flux passage from an external magnetic field through the magnetic core to yield high induced voltage in response to the external magnetic field.

Example 11. The method according to Example 10 wherein the applying of the 3D printing includes:
(a) providing curable resins corresponding respectively to the at least three materials, whereby the curable resins include:
   magnetic material resin including particles of magnetic material suspended in curable polymer binder;
conductive material resin including particles of conductive material suspended in curable polymer binder;
   non-magnetic dielectric material resin including particles of non-magnetic dielectric material suspended in curable polymer binder; and
(b) 3D printing the curable resins layer by layer according to the three-dimensional spatial distribution of the model to obtain one or more 3D structures with the one or more electric components, wherein 3D printing each layer includes:
   printing and curing the magnetic material at regions of the layer corresponding to one or more magnetic cores of the one or more electric coils;
   printing and curing the conductive material at regions of the layer corresponding to one or more inductors of the one or more electric coils; and
   printing and curing the of non-magnetic dielectric material at least near interfaces of the conductive material of the one or more inductors not interfacing the one or more magnetic cores; and
   wherein the 3D printing carried out such that the magnetic core of the at least one coil of the open magnetic circuit configuration occupies the central region of the coil/helical geometry of the inductor while not enclosing the conductive windings of the inductor with a continuous closed loop channel of the magnetic material; and
(c) sintering the one or more 3D structures at temperature sufficient for burning or driving off polymers therefrom and thereby achieving ceramic or metal density approaching 100%.

Example 12. The method according to Example 10 wherein the 3D printing is carried out with successive printing of layers of thicknesses in the order of 5 to 15 μm along a printing direction (z) and such that a pitch of the conductive windings along the printing direction (z) is in the order twice the layer thicknesses of about 10 μm to 30 μm, thereby yielding printing of miniature electric coils having high winding density.

Example 13. The method according to Example 10 suitable for mass-production of electronic components and wherein the model is indicative of material distribution in an arrangement of a plurality of the electric components to be simultaneously 3D printed.

Example 14. The method according to Example 10 wherein boundaries of the bulk are printed with one or more materials other than the conductive material such that the inductor is fully embedded within a bulk of its respective electronic component and not exposed at external surfaces of the bulks except from terminal end electric contacts thereof.

Example 15. The method according to Example 14 wherein at least one of the following:
the one or more materials printed in the boundaries of the bulk include the non-magnetic dielectric material; and
the one or more materials printed in the boundaries of the bulk include the non-magnetic dielectric material, and the magnetic material and wherein the non-magnetic dielectric material is interposed between separate regions of the magnetic material at the boundaries so as to prevent formation of a closed loop magnetic channel enclosing the conductive windings of the inductor.

Example 16. The method according to Example 10 wherein the 3D printing is carried out utilizing vat photopolymerization 3D printing process.

Example 17. The method according to Example 10 wherein at least one of the following:
the conductive material includes Silver, and the method includes sintering the one or more 3D structures at temperatures below 961° C.;
the conductive material includes Copper, and the method includes sintering the one or more 3D structures in Oxygen deprived environment (such as Nitrogen rich environment) at temperatures below 1084° C.;
the conductive material includes Silver-Palladium alloy, and the method includes sintering the one or more 3D structures at temperatures below a melting point of the Silver-Palladium alloy;
the magnetic material includes Ceramic-Ferrite material;
the magnetic material includes Metal material;
the magnetic material has a relative permeability μᵣ in the order of 100 or more;
the non-magnetic dielectric material includes Ceramic or Glass-Ceramic material and wherein the method includes sintering the one or more 3D structures at temperature sufficient for firing the Ceramic or Glass-Ceramic material.

Example 18. The method according to Example 10 further comprising furnishing contact elements connected to the electric channel defining the inductor of the at least one electric coil, at terminal regions at which the conductive material of the electric channel is exposed from the bulk, to thereby enable mounting the at least one electric coil to a circuit board.

Example 19. A magnetic sensor including one or more electric coils, the magnetic sensor is formed by 3D printing of at least three different 3D-printed materials, and includes:
a bulk formed by non-magnetic dielectric 3D printed material; and
at least one electric coil in the bulk;
wherein the at least one electric coil includes:
   at least one magnetic channel comprising 3D-printed magnetic material embedded in the bulk; and
   at least one electric channel comprising 3D-printed conductive material embedded in the bulk and extending therethrough between two terminal ends of the electric channel of the electric coil, which are exposed at a surface of the bulk to serve as electric contacts of the electric coil;
   wherein at least one of the electric channel and magnetic channel is 3D printed in the bulk with coiled geometry forming a plurality of windings about the other one of the electric channel and magnetic channel, thereby providing that the at least one electric channel is configured and operable as an inductor of the at least one electric coil and the at least one magnetic channel is configured and operable as a magnetic core of the inductor of the at least one electric coil;
   wherein the magnetic channel forming the magnetic core is configured with an open magnetic circuit configuration such that it does not enclose the conductive channel with a closed loop of the magnetic material, thereby facilitates efficient coupling of flux passage from an external magnetic field through the magnetic core and enables high induced voltage in the at least one electric coil in response to the external magnetic field.

Example 20. A method to fabricate one or more magnetic sensors including one or more electric coils, wherein at least one electric coil of the one or more electric coils has an open magnetic circuit configuration; the method includes:
providing a model of the one or more magnetic sensors wherein the model is indicative of three-dimensional spatial distribution of at least three materials in the at least one electric coil having the open magnetic circuit configuration, including: 3D-distribution of magnetic material, 3D-distribution of conductive material, and 3D-distribution of non-magnetic dielectric material;
wherein the three-dimensional spatial distribution of the at least three materials is indicative of:
   spatial distribution of a bulk of the at least one electric coil formed by non-magnetic dielectric material;
   spatial distribution of the magnetic material, defining at least one magnetic channel in the bulk associated with a magnetic core of the at least one electric coil;
   spatial distribution of the conductive material defining at least one electric channel in the bulk associated with an inductor of the at least one electric coil;
   wherein at least one of the spatial distribution of magnetic material defining the magnetic channel of the at least one electric coil, and the spatial distribution of conductive material defining the electric channel of the at least one electric coil has coiled geometry forming a plurality of windings about the other one of the electric channel and magnetic channel, thereby providing that the at least one electric channel is configured and operable as an inductor of the at least one electric coil and the at least one magnetic channel is configured and operable as a magnetic core of the inductor of the at least one electric coil;
   wherein the spatial distribution of the magnetic material defining the magnetic channel has the open magnetic circuit configuration and does not enclose the conductive channel with a closed loop of the magnetic material; and
   applying 3D printing to print the one or more magnetic sensors thereby obtaining the at least one electric coil with the open magnetic circuit configuration facilitating efficient coupling of flux passage from an external magnetic field through the magnetic core to yield high induced voltage in response to the external magnetic field.

1. An electric component comprising one or more electric coils, the electric component is formed by 3D printing of at least three different 3D-printed materials with spatial distributions yielding an open magnetic circuit configuration of at least one electric coil of said one or more electric coils; and wherein the electric component comprises:
a bulk formed by non-magnetic and dielectric 3D printed material; and
at least one electric coil of the open magnetic circuit configuration, 3D-printed in said bulk, comprising:
at least one magnetic channel comprising magnetic material 3D-printed in said bulk forming a magnetic core of the at least one electric coil; and
at least one electric channel forming an inductor of said at least one electric coil comprising conductive material 3D-printed in said bulk with a coil/helical geometry having a plurality of electrically connected conductive windings arranged to circumference the magnetic channel of the magnetic core;
wherein the magnetic channel of the magnetic core is configured and operable with said open magnetic circuit configuration and comprises magnetic material occupying a central region of the coil/helical geometry of the inductor while not enclosing said conductive windings with a closed loop of said magnetic material.

2. The electric component of claim 1 wherein said electrically connected conductive windings comprise conductive windings distributed at different 3D printed layers of said electric component thereby forming a helical arrangement of electrically connected conductive windings.

3. The electric component of claim 2 wherein a thickness of said 3D printed layers is in the order of 5 to 15 μm thereby yielding a pitch of said helical arrangement of conductive windings in the order of twice the pitch of said 3D printed layers being about 10 μm to 30 μm along a printing direction of said 3D printed layers.

4. The electric component of claim 1 wherein said electrically connected conductive windings comprise plurality of conductive windings arranged concentrically in one or more 3D printed layers of said electric component thereby forming a spiral arrangement of conductive windings in said the one or more 3D printed layers.

5. The electric component of claim 4 wherein a resolution of said 3D printed layers, after said sintering, is in the order of 15 to 30 μm, thereby yielding a pitch of said spiral arrangement of conductive windings in the order of twice the resolution of said 3D printed layers with respect to a lateral plane of said 3D printed layers.

6. The electric component of claim 5 wherein said electrically connected conductive windings comprise a plurality of said spiral arrangement of conductive windings distributed at different 3D printed layers of said bulk and electrically connected between them to form a spiral-helical arrangement of said electrically connected conductive windings.

7. The electric component of claim 1, wherein spacings between adjacent windings of said a plurality of electrically connected conductive windings are occupied by 3D printed non-electrically-conductive material being one of said non-magnetic dielectric material and said 3D-printed magnetic material.

8. The electric component of claim 1, wherein the 3D-printed conductive material of the inductor is fully embedded within said bulk and not exposed at external surfaces of said bulk, so as to isolate the inductor from environmental conditions which might degrade its physical/conductive properties.

9. The electric component of claim 1 wherein at least one of the following:
said conductive material comprises Silver;
said conductive material comprises Copper;
said conductive material comprises Silver-Palladium alloy;
said magnetic material comprises Ceramic-Ferrite material;
said magnetic material comprises Metal Material;
said magnetic material is a soft magnetic material having relative permeability μᵣ in the order of 100 or more
said non-magnetic dielectric material comprises ceramic or glass-ceramic material.

10. A method to fabricate one or more electric components comprising one or more electric coils;
wherein at least one electric coil of said one or more electric coils has an open magnetic circuit configuration; the method comprising:
providing a model of the one or more electric components wherein the model is indicative of three-dimensional spatial distribution of at least three materials in the at least one electric coil having the open magnetic circuit configuration, including: 3D-distribution of magnetic material, 3D-distribution of conductive material, and 3D-distribution of non-magnetic dielectric material;
wherein the three-dimensional spatial distribution of the at least three materials is indicative of:
spatial distribution of a bulk of the at least one electric coil formed by non-magnetic dielectric material;
spatial distribution of magnetic material, defining at least one magnetic channel in said bulk associated with a magnetic core of the at least one electric coil;
spatial distribution of conductive material defining at least one electric channel in said bulk associated with an inductor of said at least one electric coil and having a coiled/helical geometry with a plurality of electrically connected conductive windings arranged to circumference said magnetic channel;
wherein the spatial distribution of the magnetic material has said open magnetic circuit configuration and is indicative of magnetic material occupying a central region of the coil/helical geometry of said inductor while not enclosing the conductive windings of said inductor with a closed loop of said magnetic material; and
applying 3D printing to print said one or more electric components thereby obtaining said at least one electric coil with the open magnetic circuit configuration facilitating efficient coupling of flux passage from an external magnetic field through the magnetic core to yield high induced voltage in response to the external magnetic field.

11. The method of claim 10 wherein said applying of the 3D printing comprises
(a) providing curable resins corresponding respectively to said at least three materials, whereby said curable resins comprise:
magnetic material resin comprising particles of magnetic material suspended in curable polymer binder;
conductive material resin comprising particles of conductive material suspended in curable polymer binder;
non-magnetic dielectric material resin comprising particles of non-magnetic dielectric material suspended in curable polymer binder; and
(b) 3D printing said curable resins layer by layer according to said three-dimensional spatial distribution of the model to obtain one or more 3D structures with said one or more electric components, wherein 3D printing each layer comprises:
printing and curing said magnetic material at regions of said layer corresponding to one or more magnetic cores of said one or more electric coils;
printing and curing said conductive material at regions of said layer corresponding to one or more inductors of said one or more electric coils; and
printing and curing said of non-magnetic dielectric material at least near interfaces of said conductive material of the one or more inductors not interfacing said one or more magnetic cores; and
wherein said 3D printing carried out such that the magnetic core of said at least one coil of the open magnetic circuit configuration occupies the central region of the coil/helical geometry of said inductor while not enclosing the conductive windings of said inductor with a continuous closed loop channel of said magnetic material; and
(c) sintering said one or more 3D structures at temperature sufficient for burning or driving off polymers therefrom and thereby achieving ceramic or metal density approaching 100% in said electric components.

12. The method of claim 10 wherein said 3D printing is carried out with successive printing of layers of thicknesses in the order of 5 to 15 μm along a printing direction (z) and such that a pitch of the conductive windings along said printing direction (z) is in the order twice said layer thicknesses of about 10 μm to 30 μm thereby yielding printing of miniature electric coils having high winding density.

13. The method of claim 10 suitable for mass-production of electronic components and wherein said model is indicative of material distribution in an arrangement of a plurality of said electric components to be simultaneously 3D printed.

14. The method of claim 10 wherein boundaries of said bulk are printed with one or more materials other than said conductive material such that said inductor is fully embedded within a bulk of its respective electronic component and not exposed at external surfaces of said bulks except from terminal end electric contacts thereof.

15. The method of claim 14 wherein at least one of the following:
said one or more materials printed in the boundaries of said bulk comprise said non-magnetic dielectric material; and
said one or more materials printed in the boundaries of said bulk comprise said non-magnetic dielectric material, and said magnetic material and wherein said non-magnetic dielectric material is interposed between separate regions of said magnetic material at said boundaries so as to prevent formation of a closed loop magnetic channel enclosing the conductive windings of the inductor.

16. The method of claim 10 wherein said 3D printing is carried out utilizing vat photopolymerization 3D printing process.

17. The method of claim 10 wherein at least one of the following:
said conductive material comprises Silver, and the method comprises sintering said one or more 3D structures at temperatures below 961° C.;
said conductive material comprises Copper, and the method comprises sintering said one or more 3D structures in Oxygen deprived environment (such as Nitrogen rich environment) at temperatures below 1084° C.;
said conductive material comprises Silver-Palladium alloy, and the method comprises sintering said one or more 3D structures at temperatures below a melting point of the Silver-Palladium alloy;
said magnetic material comprises Ceramic-Ferrite material;
said magnetic material comprises Metal material;
said magnetic material has a relative permeability μᵣ in the order of 100 or more;
said non-magnetic dielectric material comprises Ceramic or Glass-Ceramic material and wherein the method comprises sintering said one or more 3D structures at temperature sufficient for firing said Ceramic or Glass-Ceramic material.

18. The method of claim 10 further comprising furnishing contact elements connected to the electric channel defining the inductor of said at least one electric coil, at terminal regions at which the conductive material of said electric channel is exposed from said bulk, to thereby enable mounting said at least one electric coil to a circuit board.

19. A magnetic sensor comprising one or more electric coils,
the magnetic sensor is formed by 3D printing of at least three different 3D-printed materials, and comprises:
a bulk formed by non-magnetic dielectric 3D printed material; and
at least one electric coil in said bulk, whereby the at least one electric coil comprises:
at least one magnetic channel comprising 3D-printed magnetic material embedded in said bulk; and
at least one electric channel comprising 3D-printed conductive material embedded in said bulk and extending therethrough between two terminal ends of the electric channel of said electric coil, which are exposed at a surface of the bulk to serve as electric contacts of said electric coil;
wherein at least one of said electric channel and magnetic channel is 3D printed in said bulk with coiled geometry forming a plurality of windings about the other one of said electric channel and magnetic channel, thereby providing that said at least one electric channel is configured and operable as an inductor of said at least one electric coil and said at least one magnetic channel is configured and operable as a magnetic core of the inductor of the at least one electric coil;
wherein the magnetic channel forming the magnetic core is configured with an open magnetic circuit configuration such that it does not enclose said conductive channel with a closed loop of said magnetic material and thereby facilitates efficient coupling of flux passage from an external magnetic field through the magnetic core and enables high induced voltage in said at least one electric coil in response to the external magnetic field.

20. A method to fabricate one or more magnetic sensors comprising one or more electric coils;
wherein at least one electric coil of said one or more electric coils has an open magnetic circuit configuration;
the method comprising:
providing a model of the one or more magnetic sensors wherein the model is indicative of three-dimensional spatial distribution of at least three materials in the at least one electric coil with the open magnetic circuit configuration, including: 3D-distribution of magnetic material, 3D-distribution of conductive material, and 3D-distribution of non-magnetic dielectric material;
wherein the three-dimensional spatial distribution of the at least three materials is indicative of:
spatial distribution of a bulk of the at least one electric coil formed by non-magnetic dielectric material;
spatial distribution of the magnetic material, defining at least one magnetic channel in said bulk associated with a magnetic core of the at least one electric coil;
spatial distribution of the conductive material defining at least one electric channel in said bulk associated with an inductor of said at least one electric coil;
wherein at least one of the spatial distribution of magnetic material defining said magnetic channel of the at least one electric coil, and the spatial distribution of conductive material defining said electric channel of the at least one electric coil has coiled geometry forming a plurality of windings about the other one of said electric channel and magnetic channel, thereby providing that said at least one electric channel is configured and operable as an inductor of said at least one electric coil and said at least one magnetic channel is configured and operable as a magnetic core of the inductor of the at least one electric coil;
wherein the spatial distribution of the magnetic material defining said magnetic channel has said open magnetic circuit configuration and does not enclose said conductive channel with a closed loop of said magnetic material; and
applying 3D printing to print said one or more magnetic sensors thereby obtaining said at least one electric coil with the open magnetic circuit configuration facilitating efficient coupling of flux passage from an external magnetic field through the magnetic core to yield high induced voltage in response to the external magnetic field.
